(19) United States
(12) Patent Application Publication
(10) Pub. No.: US 2011/0003901 A1
(43) Pub. Date: Jan. 6, 2011

(54) FT WATER TREATING AND RECOVERY

(75) Inventor: Randall Boyd Pruet, Ottershaw (GB)

Correspondence Address: CHEVRON CORPORATION, P.O. BOX 6006, SAN RAMON, CA, 94583-0806, US

(73) Assignee: Chevron U.S.A. Inc.

(21) Appl. No.: 12/649,848

(22) Filed: Dec. 30, 2009

Related U.S. Application Data
(60) Provisional application No. 61/141,355, filed on Dec. 30, 2008.

(51) Int. Cl. C07C 1/00

(52) U.S. Cl. 518/705

(57) ABSTRACT

A process of using super critical water in a Fischer-Tropsch (FT) synthesis gas process to enhance carbon efficiency and/or process waste streams and/or provide for CO₂ recycle is described.

This application claims priority to Provisional Application 61/141,355 filed Dec. 30, 2008, the specification of which is incorporated herein by reference.

### BACKGROUND OF THE INVENTION

FT processes are concerned with carbon and thermal efficiencies. Feed carbon that is processed to CO₂ and released to the environment detracts from carbon efficiency, for example CO₂ vented at the end of the FT process.

Hydrocarbon laden reaction water from an FT reactor must be processed before releasing the water for other uses this creates additional processing costs. Sometimes a portion of the hydrocarbon is removed from the water prior to final treatment in a bio treating step this also adds costs. Sometimes the entire aqueous stream is combusted in a thermal oxidizer to remove contaminants, recover heat and return vaporous water to the environment which also adds costs to the process and lowers overall efficiency. It would be desirable to more effectively use the created reaction water and also be able to process the heaviest hydrocarbons from the reaction.

### DESCRIPTION OF THE INVENTION FIGURES AND EMBODIMENTS

The invention provides a process for treating the FT reaction water to make a pure liquid state, while providing for the processing of organic acid to be converted back to CO feed gas, or while providing for the processing of organic acid and other oxygenated hydrocarbons to be converted back to CO feed gas, and/or while providing for the processing of CO₂ to be converted back to CO feed gas, while improving the heat integration and recovery of the facility, while providing an opportunity to process contaminated wax or spent catalyst into an inert, clean and reclaimable (reusable) state. Optionally and additionally a portion or all of the heaviest portions of the created hydrocarbon stream can be treated to make more readily useable lower molecular weight products, e.g. diesel.

Benefits of the process, alone or in combination are extremely clean water that can be used for boiler feed water, CT makeup or other. If optional processing of the hydrocarbons, preferably the heaviest hydrocarbons, is used then more diesel yield can be achieved. Heaviest hydrocarbons are intended to include those having a carbon number greater than C₄₀ preferably greater than C₅₀. The process that can be used to process high or low BTU waste or waste waters. The overall FT process was improved carbon efficiency due to aqueous organics and recovered wax that are converted to CO and recycled to FT as feed and CO₂ that can be shifted to CO.

Additionally extremely clean de-waxed catalyst suitable for packaging and transportation to a reclamation location is a by product. Improved heat recovery and integration; including “steam” that is sufficiently high in pressure/temperature to recycle back to the ATR (with or without the CO) and/or HP steam that can be used in a variety of ways as well as the potential to reduce CO₂ emissions from an FT process.

The important element of this process is the supercritical water partial (or complete) oxidation (SCWPO) reactor. SCWPO involves carrying out oxidative reactions in a supercritical water environment—akin to high-pressure steam—in the presence of substoichiometric quantities of oxidant, typically pure oxygen or air. Any source of water may be used in the fluid comprising water in practicing the present invention. Sources of water include but are not limited to drinking water, treated or untreated wastewater, river water, lake water, seawater produced water or the like. The water produced during the Fischer-Tropsch (FT) process is an ideal source of water because it is formed in amounts greater than 50% of the product species of the FT reaction.

In accordance with the invention, the hydrocarbon feed and the fluid comprising water are contacted in a mixing zone prior to entering the reaction zones. In accordance with the invention, mixing may be accomplished in many ways and is preferably accomplished by a technique that does not employ mechanical moving parts. Such means of mixing may include, but are not limited to, use of static mixers, spray nozzles, sonic or ultrasonic agitation. The oil and water should be heated and mixed so that the combined stream will reach supercritical water conditions in the reaction zone. Various heating sequences may be employed to accomplish this result.

For example, oil and water streams may be heated separately at different temperatures so that supercritical water mixes with and heats the remaining oil so that the entire stream will reach supercritical conditions in the reaction zone. Other methods of mixing and heating sequences may be used as will be recognized by those skilled in the art.

After the reactants have been mixed, they are passed into a reaction zone in which they are allowed to react under temperature and pressure conditions of supercritical water, i.e. supercritical water conditions, in the absence of externally added hydrogen, for a residence time sufficient to allow upgrading reactions to occur. The reaction is preferably allowed to occur in the absence of externally added catalysts or promoters, although the use of such catalysts and promoters is permissible in accordance with the present invention.

“Hydrogen” as used herein in the phrase, “in the absence of externally added hydrogen” means hydrogen gas. This phrase is not intended to exclude all sources of hydrogen that are available as reactants. Other molecules such as saturated hydrocarbons may act as a hydrogen source during the reaction by donating hydrogen to other unsaturated hydrocarbons. In addition, H₂ may be formed in-situ during the reaction through steam reforming of hydrocarbons and water-gas-shift reaction.

The reaction zone preferably comprises a reactor, which is equipped with a means for collecting the reaction products (syncrude, water, and gases such as H₂ and CO), and a bottom section where any metals or solids (the “dreg stream”) may accumulate. Supercritical water conditions include a temperature from 374° C. (the critical temperature of water) to 1000° C., preferably from 374° C. to 600° C. and most preferably from the 374° C. to 400° C., a pressure from 3,205 (the critical pressure of water) to 10,000 psia, preferably from 3,205 psia to 7,200 psia and most preferably from 3,205 to 4,000 psia, an oil/water volume ratio from 1:0.1 to 1:10, preferably from 1:0.5 to 1:3 and most preferably about 1:1 to 1:2.

The reactants are allowed to react under these conditions for a sufficient time to allow upgrading reactions to occur. Preferably, the residence time will be selected to allow the upgrading reactions to occur selectively and to the fullest extent without having undesirable side reactions of coking or residue formation. Reactor residence times may be from 1 minute to 6 hours, preferably from 8 minutes to 2 hours and most preferably from 20 to 40 minutes.

After the reaction has progressed sufficiently, a single phase reaction product is withdrawn from the reaction zone, cooled, and separated into gas, effluent water, and upgraded hydrocarbon phases. This separation is preferably done by cooling the stream and using one or more two-phase separators, three-phase separators, or other gas-oil-water separation device known in the art. However, any method of separation can be used in accordance with the invention.

The composition of gaseous product obtained by treatment of the heavy hydrocarbons in accordance with the process of the present invention will depend on feed properties and typically comprises light hydrocarbons, water vapor, acid gas (CO₂ and H₂S), methane and hydrogen. The effluent water may be used, reused or discarded. It may be recycled to e.g. the feed water tank, the feed water treatment system, to the reaction zone, or used as reactor cooling water for the Fischer-Tropsch reaction.

Any upgraded hydrocarbon product, which is sometimes referred to as “syncrude” herein may be upgraded further or processed into other hydrocarbon products using methods that are known in the hydrocarbon processing art.

The process of the present invention may be carried out as a continuous or semi-continuous process or a batch process. In the continuous process the entire system operates with a feed stream of oil and a separate feed stream of supercritical water and reaches a steady state; whereby all the flow rates, temperatures, pressures, and composition of the inlet, outlet, and recycle streams do not vary appreciably with time.

While not being bound to any theory of operation, it is believed that a number of upgrading reactions are occurring simultaneously at the supercritical reaction conditions used in the present process.

In a preferred embodiment of the invention the major chemical/upgrading reactions are believed to be:

Thermal Cracking: CₓH_{y}→lighter hydrocarbons
Steam Reforming: CₓH_{y}+2xH₂O=xCO₂+(2x+y/2)H₂

### Water-Gas-Shift: CO+H₂O═CO₂+H₂

Demetalization: CₓH_{y}Ni_{w}+H₂O/H₂→NiO/Ni(OH)₂+lighter hydrocarbons
Desulfurization: CₓH_{y}S_{z}+H₂0/H₂═H₂S+lighter hydrocarbons

The exact pathway may depend on the reactor operating conditions (temperature, pressure, O/W volume ratio), reactor design (mode of contact/mixing, sequence of heating), and the hydrocarbon feedstock.

The SCWPO aspect and reactor of the invention is additionally discussed in the US2008/0099376; US2008/0099378; US2008/0099374; U.S. Ser. No. 11/906,852 now US2009/0089921; U.S. Ser. No. 11/966,852 now US2009/0166262; U.S. Ser. No. 11/966, 708 now US2009/0166261; and U.S. Ser. No. 11/960,891 now US2009/0159498 the teachings of specifications of each are incorporated herein by reference for all purposes.

A major advantage is that the high-pressure, high-density aqueous environment is ideal for reacting and gasifying organics. The high water content of the medium should encourage formation of hydrogen and hydrogen-rich products and is highly compatible with high water content feeds. Also, the high water content of the medium is effective for gasification of hydrogen-poor materials. Supercritical water (SCW) gasification and partial oxidation technology is based on the unique properties of water at conditions near and beyond its thermodynamic critical point of 705° F. and 3206 psia. At typical SCW reactor conditions of 1200° F. and 3400 psi densities are only one-tenth that of normal liquid water. Hydrogen bonding is almost entirely disrupted, so that the water molecules lose the ordering responsible for many of liquid water's characteristic properties. In particular, solubility behavior is closer to that of high-pressure steam than to liquid water. The loss of bulk polarity by the water phase has striking effects on normally water-soluble salts. No longer readily solvated by water molecules, they frequently precipitate out as solids. Small polar and nonpolar organic compounds, with relatively high volatility, will exist as vapors at typical SCW conditions, and hence will be completely miscible with supercritical water. Gases such as N₂, O₂, and CO₂ show similar complete miscibility. Larger organic compounds and polymers will hydrolyze to smaller molecules at typical SCW conditions, thus resulting in solubilization via chemical reaction. Benefits of a super critical partial or complete oxidation reactor in various embodiments are at least one or more of the following:

Does not require pretreating, feeds or a feed storage tank. Does not consume steam. Generates boiler feed water quality water or steam which can be directly returned as makeup water used for power generation or in steam reforming to form syngas for the Fischer-Tropsch reaction. Cleans wax off catalyst prior to shipping. Processes contaminated wax. Can accept other oily or sour water, other offspec hydrocarbon. Consumes O₂ to provide CO and H₂ synthesis gas. Operates at high pressure and temperature so produced steam, CO and H₂ may be extracted at choice of pressures and temperatures and reintroduced to process. Generates high pressure (HHP) steam with gases, could be used to generate power. Requires HHP feed pump. Converts nitrogen compounds e.g. ammonia to nitrous oxide. Process bench scale are available for testing reaction water, catalyst, etc.

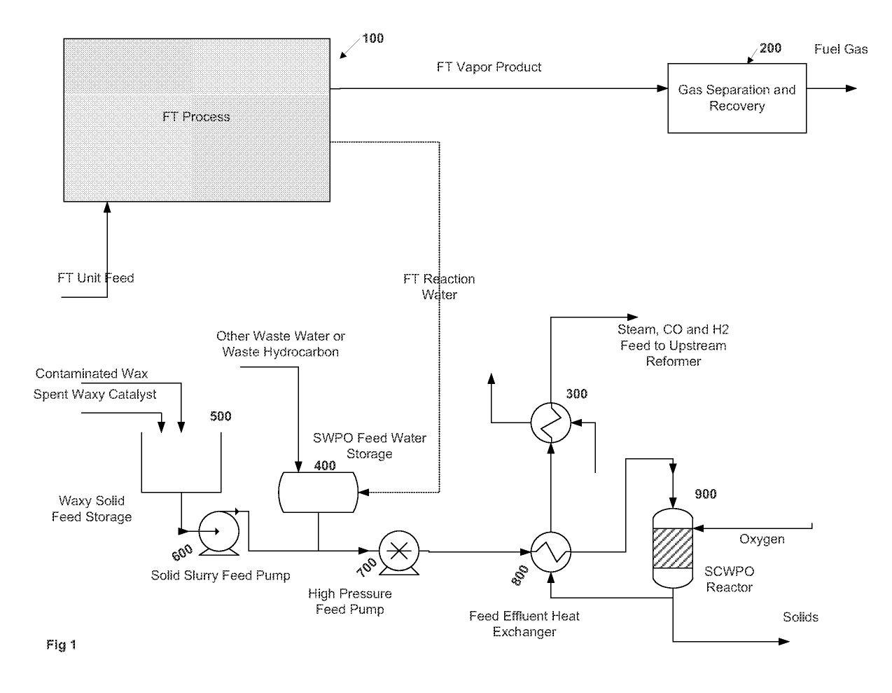

FIG. 1 Process embodiment using SCWPO feed to an Auto thermal reformer

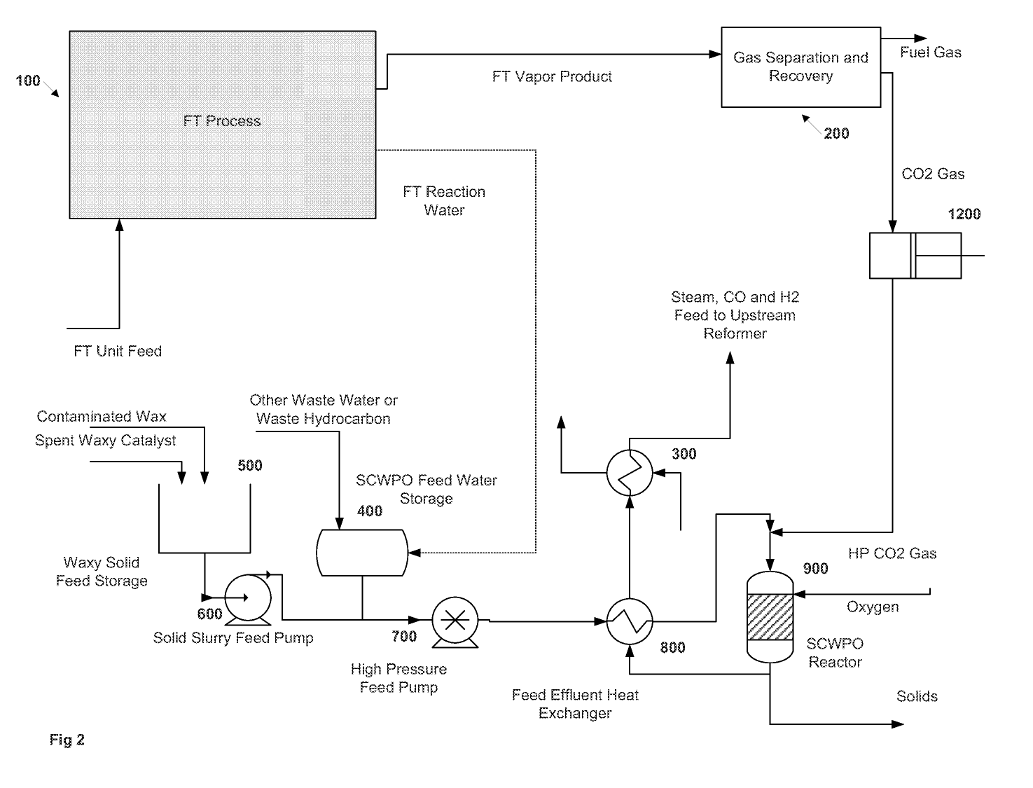

FIG. 2 Process embodiment using SCWPO feed to upstream reformer CO₂ recycle

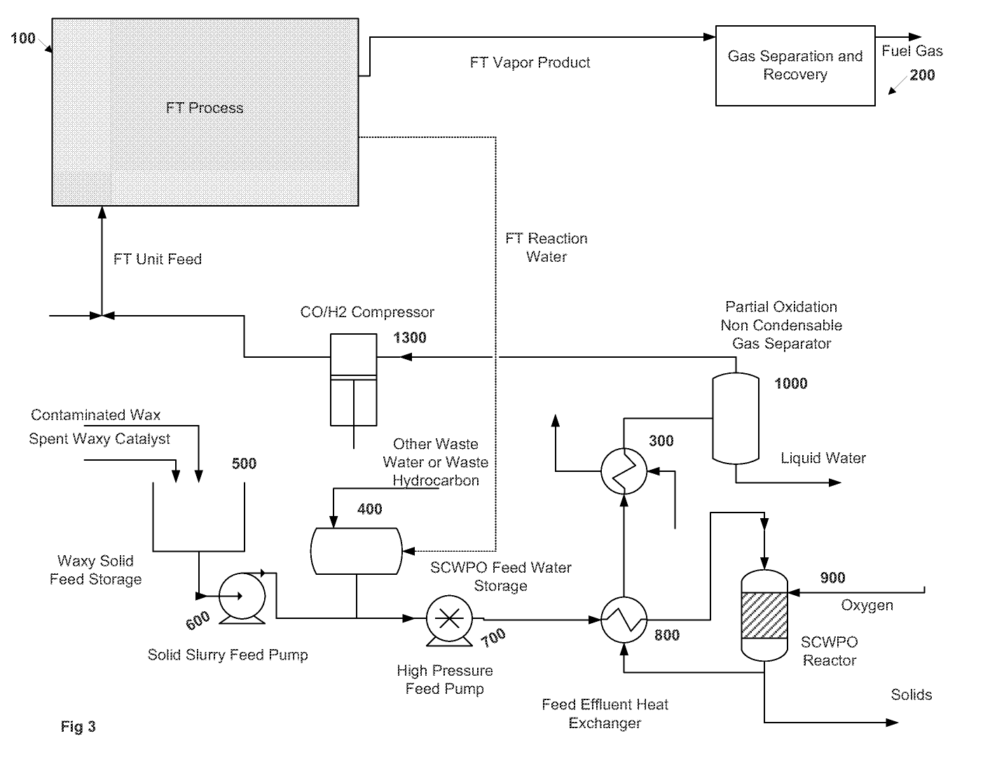

FIG. 3 Process embodiment using SCWPO-FT to feed the FT reactor

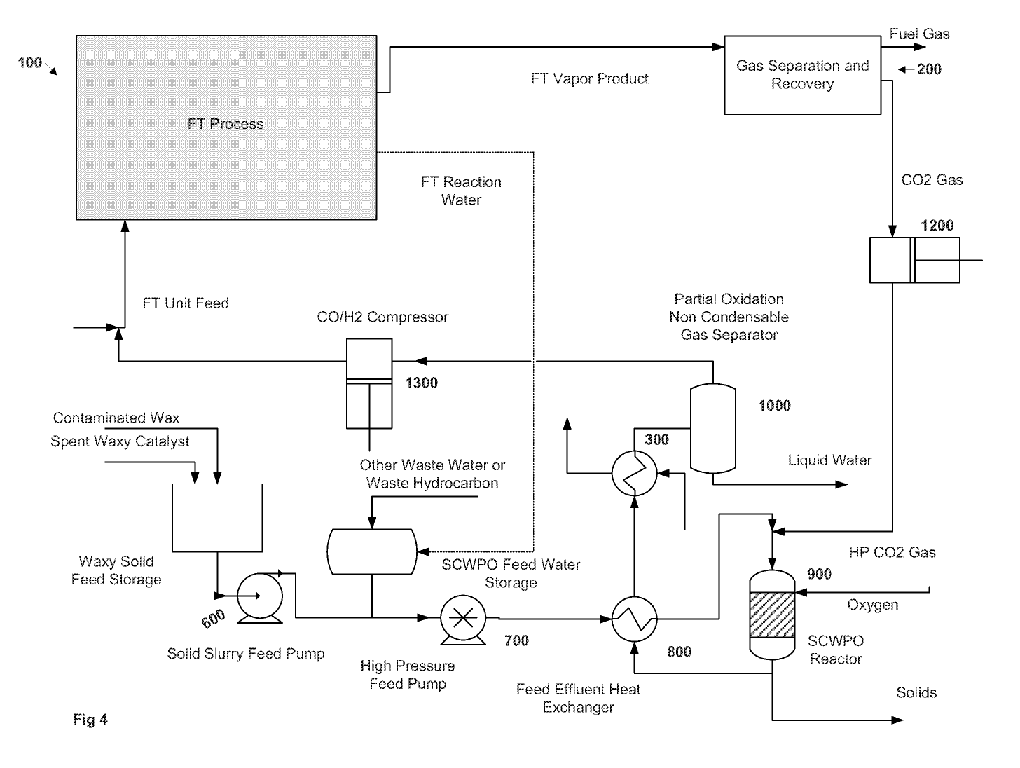

FIG. 4 Process embodiment using SCWPO to upstream reformer with CO₂ recycle

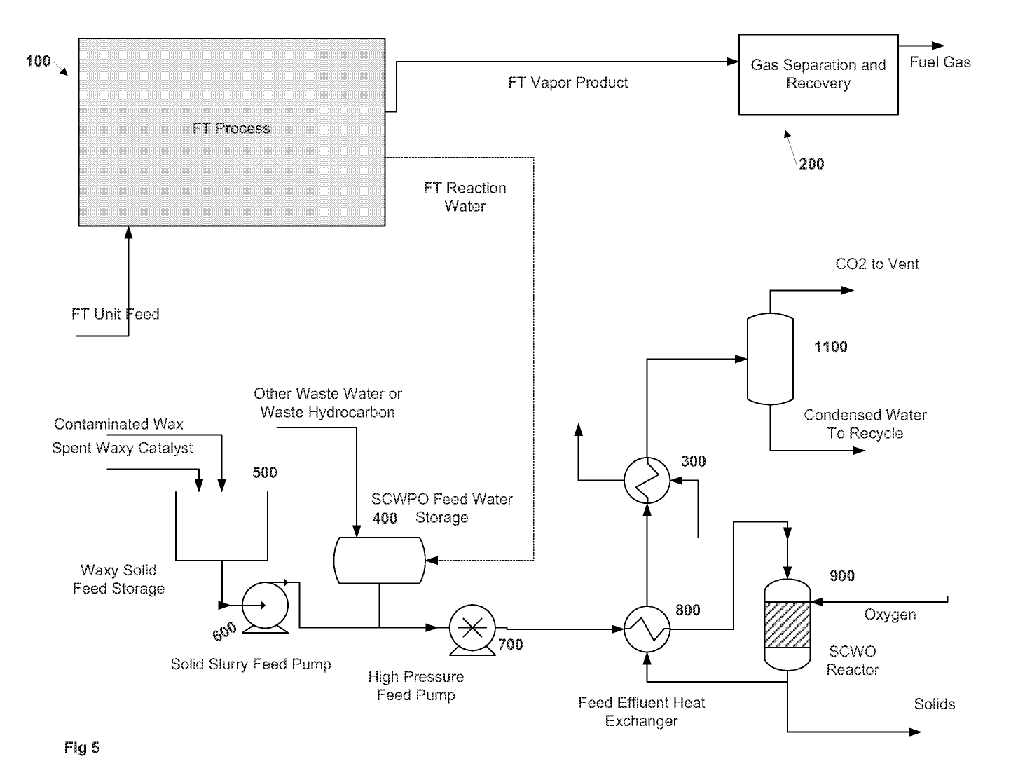

FIG. 5 Process embodiment using SCWPO complete to CO₂ in FT reactor

Turning now to FIGS. 1 through 5, common elements in each embodiment are numbered the same. The process includes an Upstream Fischer-Tropsch FT Process 100 and Gas Recovery Unit 200 additionally, Heat Exchangers 300 and 800 with the Super Critical Water Reactor 900 are included in all embodiments. FT Reaction Water is fed to a Feed Storage Unit 400 in each embodiment. All embodiments contain a Contaminated Wax and Spent Slurry unit with a solid slurry feed pump and a high pressure feed pump through the heat exchanger to the super critical water reactor.

FIG. 3 further includes a partial oxidation non condensable gas separator in communication with Heat Exchanger 300 and a CO₂/H₂Compressor 1300. Embodiment 3 includes a gas liquid separator to vent the gases such as CO₂ and a condenser to recycle the water. FIG. 2 includes a CO₂ compressor 1200 to feed high pressure CO₂ to the super critical water reactor to precipitate out solids. The last embodiment in FIG. 5 includes the CO₂ gas compressor 1200 the partial oxidation Non Condensable Gas Separator 1000 in communication with the CO₂ Compressor 1300. The benefits of these embodiments are described herein after.

In this embodiment the reactor is configured to accept reaction water directly from the FT process or reaction water that has been processed to reduced hydrocarbon content. In the SCWPO reactor aqueous hydrocarbon is oxidized to produce as much as 80% CO/H₂ (1.25/1 ratio) with remainder CO₂ and CH₄. Also an aqueous slurry of contaminated wax or wax contaminated spent catalyst can be fed to the reactor. Precious metals from the catalyst exit the reactor for subsequent reclamation. Referring to the figures, there are a number of ways the system can be configured dependent on the desired results. For example Case 1 depicts heat recovery only to the extent required to match effluent conditions to upstream reformer operating conditions. In this case all gases and steam are recycled back to the upstream reformer. FIG. 3 on the other hand depicts the maximum heat recovery for partial oxidation with complete condensation of the effluent water. The non-condensable gases would be compressed and recycled directly to the FT reactor. FIG. 5 depicts the complete combustion of the hydrocarbon feed to CO₂ with a maximum heat recovery and a liquid water outlet suitable for boiler feed water makeup. FIG. 2 is an enhancement of FIG. 1 depicting other potentially CO₂ rich streams recycled to the SCWPO feed. In the same way FIG. 4 uses additional feed gas rich in CO₂.

The amount of pure oxygen to the SCWPO reactor is controlled (based on hydrocarbon feed content) to produce either a partially oxidized product high in CO/H₂ or a completely oxidized product (CO₂) with water as the primary product.

Operating conditions, i.e. SCWPO pressure and temperature, can be adjusted to meet the effluent composition desired. The equilibrium established at the SCWPO conditions, dictates a balance of CO₂ and CO. Therefore compressed CO₂ can also be concurrently processed in the SCWPO to produce a more CO product.

Finally as the SCWPO reaction process will be a net exothermic process, energy can be extracted for beneficial use. For example although most of the reactor effluent is cooled by the reactor feed some excess energy remains in the system and can be used for a variety of purposes.

What is claimed is:

1. A Fischer-Tropsch and super critical water process which comprises:
a. reacting synthesis gas over a Fischer-Tropsch catalyst under Fischer-Tropsch conditions produce hydrocarbons and water
b. separating the water from the hydrocarbons; and
c. processing the reaction water under supercritical water processing conditions to produce reaction products which from the feed stream to the components of the Fischer-Tropsch process selected from the group consisting of the auto thermal reformer, synthesis gas components for the Fischer-Tropsch reactor, or make up cooling water for the Fischer-Tropsch reactor, or combinations thereof.

2. The process according to claim 1 wherein the supercritical water processing of the reactant water created steam, CO and H₂ for a feed to the auto thermal reformer.

3. The process according to claim 2 wherein further comprising the addition of CO₂ from a gas separator and recovery unit into the super critical water reactor.

4. The process according to claim 1 wherein the products of the super critical water reactor provide CO and H₂ feed components to the Fischer-Tropsch reactor.

5. The process according to claim 4 wherein further comprising the addition of CO₂ from a gas separator and recovery unit into the super critical water reactor.

6. The process according to claim 1 wherein the products of the super critical water reactor are condensed to produce recycle cooling water for the Fischer-Tropsch reactor.

7. In a Fischer-Tropsch process for producing hydrocarbons and water reactant products from synthesis gas over a catalyst, the improvement which comprises:
a. processing the reaction water containing hydrocarbons at super critical water process condition to form feed components for the Fischer-Tropsch process components selected from the group consisting of the auto thermal reformer, synthesis gas components for the Fischer-Tropsch reactor, make up cooling water for the Fischer-Tropsch reactor, or combinations thereof.

8. The process according to claim 7 wherein the supercritical water processing of the reactant water created steam, CO and H₂ for a feed to the auto thermal reformer.

9. The process according to claim 8 wherein further comprising the addition of CO₂ from a gas separator and recovery unit into the super critical water reactor.

10. The process according to claim 7 wherein the products of the super critical water reactor provide CO and H₂ feed components to the Fischer-Tropsch reactor.

11. The process according to claim 9 wherein further comprising the addition of CO₂ from a gas separator and recovery unit into the super critical water reactor.

12. The process according to claim 7 wherein the products of the super critical water reactor are condensed to produce recycle cooling water for the Fischer-Tropsch reactor.

13. The process according to claim 1 further including a portion of the hydrocarbons from Step a with the reaction water in Step c.

14. The process according to claim 7 further including hydrocarbons from the Fischer-Tropsch reaction in with the reaction water.
